(19) United States
(12) Patent Application Publication
(10) Pub. No.: US 2009/0275791 A1
(43) Pub. Date: Nov. 5, 2009

(54) CERAMIC FOAM CATALYST SUPPORT FOR GASOLINE ALKYLATION

(75) Inventor: Cemal Ercan, Dhahran (SA)

Correspondence Address: BRACEWELL & GIULIANI LLP, P.O. BOX 61389, HOUSTON, TX, 77208-1389, US

(73) Assignee: SAUDI ARABIAN OIL COMPANY, Dhahran (SA)

(21) Appl. No.: 12/115,071

(22) Filed: May 5, 2008

(51) Int. Cl. B01J 29/08; C07C 5/27

(52) U.S. Cl. 585/654; 502/79

(57) ABSTRACT

This invention relates to an alkylation catalyst, a method for preparing the alkylation catalyst, and a method for alkylating olefins and paraffins. The alkylation catalyst includes a foam catalyst support wherein active catalyst particles have been appended to the surface of the foam support.

### BACKGROUND OF THE INVENTION

1. Technical Field of the Invention

This invention generally relates to the field of hydroprocessing catalysts for treatment of hydrocarbons. In particular, the present invention is directed to a solid acid alkylation catalyst, a method for preparing solid acid alkylation catalysts and a method of alkylating light olefins and paraffins with a solid acid alkylation catalyst.

2. Description of the Prior Art

Alkylate is one main ingredient in reformulated gasoline. Generally, gasoline is a blend of several refinery streams, which are designed to meet the required properties of octane number, vapor pressure, etc. In modern refineries, gasoline alkylation is an important process. Almost every refinery that includes a Fluid Catalytic Cracking (FCC) unit also includes an alkylation unit. Alkylate contributes approximately 12% of the gasoline pool in North America, and is expected to contribute between 20-25% of the gasoline pool in the near future. Alkylate can be prepared from alkylation reactions involving light olefins and paraffins, thus it combines two small molecules into a valuable gasoline range molecule. Due to excellent properties, alkylate is the most desirable gasoline blending compound. Its importance continues to grow due to environmental restrictions on other blending compounds. Additionally, the phase out of MTBE (methylterbutyl ether) may also increase alkylate demand.

Alkylate is valued as a blending component because it has no olefin content, no aromatic content, low sulfur content, low vapor pressure, ideal combustion properties, and relatively high research octane numbers (RON). Alkylation processes can yield a low vapor pressure, high-octane gasoline blend stock of alkylate having between 7 and 9 carbon atoms from small, relatively cheap off-gases from the refinery. The refinery off-gases typically include olefins having between 3 and 5 carbon atoms, which through the alkylation process, can be used to create higher molecular weight alkylate, resulting in the valuable gasoline blending components described above.

Alkylation is the chemical addition of an alkyl group to another molecule to form a larger molecule. Exemplary, commercial alkylation processes include aromatic alkylation and olefin/paraffin alkylation. Aromatic alkylation processes involve the production of alkylaromatic compounds (e.g., ethylbenzene, cumene) by alkylating an aromatic compound (e.g., benzene) with an olefin (e.g., ethylene, propylene). Olefin/paraffin alkylation involves the reaction of a saturated hydrocarbon and an olefin to produce a branched saturated hydrocarbon having a higher molecular weight, such as for example, the alkylation reaction of isobutane (a saturated hydrocarbon) and 2-butene (an olefin) to produce C₈ alkylate having a high octane number.

Standard industrial alkylation processes are currently carried out in alkylation reactors that require highly concentrated liquid acid catalysts, particularly HF and H₂SO₄. Both HF and H₂SO₄ suffer from a variety of well known safety and environmental concerns, including but not limited to, high toxicity, high corrosiveness and disposal concerns. Equipment corrosion as a result of acid exposure is a major concern associated with the current processes and the use of such strong liquid acids. Therefore, there is much interest in the development and use of solid acid catalysts (SACs), specifically alkylation processes that use solid acid catalysts as substitutes in sulfuric acid or hydrofluoric acid based alkylation processes.

### SUMMARY OF THE INVENTION

An alkylation catalyst composition, a method for the preparation of the alkylation catalyst composition and a method for alkylating a hydrocarbon feedstock are provided. The alkylation catalyst composition includes at least one active metal and a support material.

In one aspect, a method for producing an alkylation catalyst is provided. The method includes the steps of providing a ceramic foam support having a surface, wherein the ceramic foam support is operable to carry activated catalytic material on the surface of the ceramic foam and ceramic foam support having a porosity of at least 75% and a pore density of at least 10 pores per inch. A coating is applied to at least a portion of the surface of the ceramic foam support such that particles of the solid acid catalyst adhere to at least a portion of the surface of the ceramic foam support to create the gasoline alkylation catalyst. The coating includes a carrier fluid and the solid acid catalyst particles, wherein the carrier fluid is suitable to maintain the particles of the solid acid catalyst in a dispersion and the solid acid catalyst has an acidity activity index of at least 1.0. In certain embodiments, the solid acid catalyst is a zeolite. In certain embodiments, the zeolite is selected from zeolite-X, zeolite-Y, zeolite beta, MCM-36 and ITQ-2. Preferably, the acid activity index is greater than 1.0.

In another aspect, a gasoline alkylation catalyst is provided. The alkylation catalyst includes a ceramic foam support, wherein the support has a porosity of greater than 70%. The surface of the ceramic foam support includes an interior surface and an exterior surface, wherein the interior surface defines a tortuous path. A solid acid catalyst having an acid activity index of greater than 1.0 is adhered to the surface of the ceramic foam. The gasoline alkylation catalyst is operable to convert alkane and olefin feed into an alkylate product. The ceramic foam support has a particle size generally in the range of 3 mm to 6 mm. In certain embodiments, the solid acid catalyst particles are selected from zeolite-X, zeolite-Y, zeolite beta, MCM-36 and ITQ-2.

In yet another aspect, a method for alkylating an alkane with an olefin is provided. The method includes the steps of introducing an alkane and olefin into a reaction zone, wherein said reaction zone includes an alkylation catalyst, to produce an alkylate product. The alkylation catalyst includes a solid acid catalyst on a ceramic foam support, wherein the ceramic foam support defines a ceramic foam support particle having an average diameter of between 3 and 6 mm. A product stream comprising the alkylate product is collected from the reaction zone. The alkane includes between 3 and 8 carbon atoms, and the olefin includes between 2 and 8 carbon atoms. The ceramic foam support has a porosity of at least 70%. In certain embodiments, the solid acid catalyst is selected from zeolite-X, zeolite-Y, zeolite beta, MCM-36 and ITQ-2. In certain embodiments, the solid acid catalyst has an acidity activity index of greater than 1.

### DETAILED DESCRIPTION OF THE INVENTION

Catalysts useful for the alkylation of olefins and paraffins to create useful gasoline blending compounds and octane enhancers and their methods of preparation and use are provided herein.

The use of acidic zeolite type materials as solid acid catalysts were previously studied in an effort to avoid the disadvantages associated with using toxic and corrosive liquid acid catalysts. However, prior art solid acid catalysts suffered from frequent pore plugging, which was often caused by large molecules which were generated during the olefin oligomerization. Plugging of the catalyst pores frequently leads to rapid deactivation of the catalyst. Additionally, because alkylation reactions are kinetically fast, over-alkylation products are frequently formed, resulting in much larger molecules. These larger molecules resulting from over-alkylation can plug the mouth of the pores, preventing subsequent catalyzation during the alkylation process, and thus stopping the reaction.

Catalysts according to the present invention are thus provided that include solid acid catalyst attached to the surface of highly porous ceramic foam support.

One exemplary support for the alkylation catalyst is ceramic foam, or like material. In general, ceramic foams are desirable as catalyst support materials because they exhibit high temperature stability, high permeability, high porosity, low pressure drop, high heat transfer and high mass transfer. Ceramic foams are most commonly used as support materials for metal melt filtration, ion-exchange filtration, heat exchangers, catalyst support, refractory linings, thermal protections systems, diesel soot traps, flame rectifiers, and solar radiation collars.

Ceramic foams catalyst supports are typically preformed retriculated structures that are positive images of plastic foams. In certain embodiments, the ceramic foams form sponge-like structures which are interconnected though openings and windows created by the ceramic struts. Preferably, the ceramic foams are highly porous. In certain embodiments, the ceramic foam can have a porosity of greater than about 60%, 70%, 75% or over 80%. In certain other embodiments, the ceramic foam can have a porosity of between about 70 and 90%. In certain preferred embodiments, the ceramic foam can have a porosity of between about 80 and 90%. The highly porous embodiments of the ceramic foam can include a plurality megapores throughout the structure. As used herein, “megapores” are defined as pores in the support that are between approximately 0.01 mm and approximately 2 mm in diameter. Preferably, the ceramic foam pores are between about 0.03 mm and about 1.5 mm in diameter, and even more preferably between about 0.05 mm and about 1.4 mm. Typically, the ceramic foam pores have a generally spherical shape, although a variety of pore shapes are possible. The high degree of porosity of the ceramic foam support provides an extensive surface area that includes exterior surface on the outside of the ceramic foam support particle, and interior surface that includes the surfaces of individual struts forming the openings and windows that define the tortuous path through the ceramic foam support material.

Large pores in the ceramic foam are useful to allow the passage of a wide variety of molecules through the support material. As noted previously, the use of supports that have a small pore size and/or lower porosity frequently results in plugging of the pores, thereby slowing or stopping the reaction. Frequent plugging of the pores leads to a reduced catalyst lifetime, thereby increasing the frequency for replacement or regeneration of the catalyst. Thus, the use of ceramic foams having large pores reduces the failure rate of the catalyst.

In certain embodiments, the pore density of ceramic foam supports can be greater than about 10 pores per inch (PPI), preferably greater than about 20 PPI, and in some embodiments, about 30 PPI or greater. In certain embodiments, the porosity is between about 10 and about 500 PPI. Preferably, porosity is in the range of about 10-80 PPI, and most preferably in the range of about 15-60 PPI. The pores generally have a high degree of interconnectivity and can be characterized by a average pore diameter dp of between about 150 μm and about 1500 μm. Preferably, dp is between about 300 μm and about 1000 μm.

In certain embodiments, the crush strength of the ceramic foam material can be preferably in the range of between about 100 and about 600 lbs/sq. inch, more preferably in the range of between approximately 250 and about 500 lbs/sq. inch.

The ceramic foam support can be prepared in any size suitable for the reactor employed. In certain embodiments, the ceramic support can be a plurality of ceramic foam support particles. In certain embodiments, the particles are between 2.5 and 10 mm in diameter. In certain embodiments, the particles are between 3.0 and 8.0 mm in diameter. In yet other preferred embodiments, the particles are between 3.0 and 6.0 mm in diameter. In certain embodiments, the ceramic foam particles can be cube shaped. In other embodiments, the ceramic foam particles can be cylindrical, square, rectangular, round or oval shaped. Preferably, the size and shape of the ceramic foam particles is such that it allows for uniform dispersions in any specific size or shape of reactor bed. Exemplary ceramic foam materials include: Al₂O₃, ZrO₂, SiC, TiO₂, mullite, Si₂N₃, and combinations thereof.

Known techniques for producing ceramic foam supports can be classified into three categories: sponge-replication, foaming agents or space holder method.

Sponge replication generally consists of using a natural sponge or polyurethane foam as the form, which can be infiltrated with a ceramic slurry. The ceramic slurry can then be fired to free the ceramic foam.

Foaming agents can be also used to create ceramic foam supports. Gas evolving constituents are added to a pre-ceramic melt. During treatment, bubbles are generated, thereby causing the material to foam. Foaming uniformity and cell geometry can be adjusted by selection of the type and amount of surfactants and foaming agents.

The space holder method is yet another method for preparing ceramic foam supports. In one example, sodium chloride is sintered and compacted to form a porous space holder, which can then be infiltrated with a polycarbosilane polymer. The salt can be dissolved using water, and polycarbosilane remains. The foam can then be pyrolyzed to form a silicon carbide (SiC) foam. Other space holders utilizing different materials can also be used to prepare ceramic foam supports.

A coating that includes catalyst particles can be applied to the surface of the ceramic foam supports, regardless of the method of preparation of the support material, by washcoating the surface of the ceramic foam or by any known technique for providing a coating.

The solid acid catalyst coating the surface of the ceramic foam support can function as the equivalent of a bed of small particles, and the use of ceramic foam supports having a catalyst applied to the surfaces generally requires smaller volume requirements as compared to an equivalent performing particle bed. Thus, catalysts that utilize ceramic foam as the support materials generally exhibit a higher catalytic activity per unit volume than the equivalent non-ceramic foam particulate catalyst material. This results an overall efficient utilization of catalytically active materials. Additionally, utilization of ceramic foam supported catalyst materials results in lower catalyst requirements, and/or less frequent regeneration/replacement of equivalent volumes of catalyst.

The high porosity and tortuosity of the ceramic foam support enhances the turbulence, mixing and transport of fluids through the support. This in turn results in significant advantages due to the use of ceramic foam supported catalysts for certain catalytic processes, especially those processes which would otherwise be limited by mass transfer or heat transfer. Additionally, the improved mixing can increase reaction rates, increase reaction yields, and reduce contact times. Furthermore, the natural mixing promoted by the ceramic foam supports can also reduce the need for mixing apparatuses or agitators.

The high porosity of ceramic foam supports also results in a lower pressure drop, particularly when compared with beds that are packed with small particles. In certain embodiments, the pressure drop is at least 3 times less than that with a packed bed. In certain other embodiments, the pressure drop can be as much as 5 times less than that with a packed bed. In yet other embodiments, the pressure drop can be up to 10 times less than that with a packed bed. The decreased pressure drop means that smaller pumps can be used with the reactor. Energy consumption and expenses for the overall process is decreased as a result of the removal or reduction in size of the various pumps necessary for the process.

As noted above, because of the tortuosity of the passages connecting the pores, the ceramic foam support has increased mixing and increased heat transfer during the alkylation reaction, as compared with both particulate beds and less porous support materials. In certain embodiments, the heat transfer is at least two times greater than a packed particulate bed. In certain embodiments, the heat transfer is at least 5 times greater than a packed particulate bed. In yet other embodiments, the heat transfer is at least seven times greater than a packed particulate bed.

A coating that includes solid acid catalyst particles can be applied to the surface of the ceramic foam support in a variety of means. The coating can be applied by known means, such as for example, washcoating, dip coating, painting, spraying, impregnation, and the like. The coating can include a carrier, which can be present as either a flow or adhesion enhancer. In certain embodiments, the carrier can be a volatile organic solvent. In certain other embodiments, the carrier can be a polymer precursor. In certain embodiments, the coating can include an active catalyst material. The coating is preferably between about 5 μm and about 500 μm thick. In certain embodiments, the catalyst can be applied to the surface of the ceramic foam support as more than one layer. In certain embodiments, wherein multiple coating layers are applied to the ceramic foam surface, the support can be calcined between the application of each individual layer. In certain other embodiments, the ceramic foam support can be heated to an elevated temperature between applications of each coating layer to increase the rate of drying. In certain instances, a pre-coating can be applied to the surface of the ceramic foam support prior to the coating step as an adhesion promoter. Exemplary pre-coatings can include gamma-alumina or ZrO₂ or sulfated ZrO₂ coated on alpha-alumina foam particles.

An alternate process for coating the ceramic foam support with zeolite crystals includes growing zeolite crystals on the ceramic foam surface. Growing zeolite crystals on various surfaces is known in the art. Zeolite crystal sizes grown on the surface of the ceramic foam can be in the range of 100 nm to 1000 nm, preferably in the range of between 200 nm and 700 nm. Growing the zeolite crystals on the ceramic foam surface is advantage for providing nanometer sized catalyst particles on the surface of the support, which provides a substantially lower limitation to pore diffusion. The effect of catalyst particle size on pore diffusion in gasoline alkylation is well established in the literature.

In another embodiment, a layer of the catalyst can be applied to the surface of the ceramic foam support structure by impregnation. Impregnation can be accomplished by known methods, including solution impregnation by a dipping technique, slurry coating.

Suitable catalysts that may be coated onto the surface of the ceramic foam support preferably have large pore size zeolites and low Si/Al ratios. Zeolites are porous crystalline materials that can be characterized by submicroscopic channels of a particular size and/or configuration. Zeolites are typically composed of aluminosilicate, but can include a wide range of other compositions. Most zeolites are hydrated alumino-silicates that have a variety of different “open” structures having sufficient size to accommodate a variety of cations. Zeolites can function as shape selective catalysts that favor certain chemical conversions within the pores in accordance with the shape or size of the molecular reactants or products. Synthetic zeolites have a variety of uses in the petrochemical and refining industries, including use as catalysts in fluid catalytic cracking and hydrocracking.

Exemplary catalysts for use in the present invention include, but are not limited to, X-zeolite, Y-zeolite and zeolite-beta. Other zeolites for use as the solid acid catalyst can include H—Y-zeolites, USY-zeolites, MCM-22, MCM-36, MCM-49, MCM-56 the MWW family of zeolites and mordenite. Zeolites ZMS-5, ZSM-11, ZSM-12, ZSM-18, ZSM-23 and ITQ-2 can also be incorporated as the catalyst, particularly when the silicon: aluminum ratio is low. Typically, the silicon:aluminum ratio is maintained below about 20. Preferably, the silicon:aluminum ratio is maintained below about 12, and even more preferably, the ratio is maintained below about 8.

Generally, the pore size of the zeolite varies between about 0.1 nm and 20 nm, although in certain embodiments, larger and smaller pores are possible. In certain embodiments, the zeolite has an average pore diameter of greater than about 1 Å. In certain other embodiments, the zeolite has an average pore diameter of greater than about 3 Å, and in certain other embodiments, the zeolite has an average pore diameter of greater than about 4 Å.

The pore volume of certain zeolite embodiments is greater than about 0.6 cm³/g, preferably greater than about 0.75 cm³/g, and more preferably greater than about 0.8 cm³/g.

In certain embodiments, the hydrogen form of the zeolite is a powerful solid state acid, and can facilitate a variety of acid catalyzed reactions, such as for example, isomerizations and alkylations. Thus, in one aspect of the present invention, the number of strong acid sites of the zeolite are maximized. In certain embodiments for use as an alkylation catalyst, the zeolite has an acidity activity index of greater than about 1.0. In certain preferred embodiments, the index is greater than about 1.2, more preferably greater than about 1.4, and most preferably at least about 1.6.

It is believed that exposure to elevated temperatures and moisture may result in the destruction of strong acid sites. Thus, in certain embodiments, exposure of the zeolite to moisture and/or high temperatures is minimized. In certain embodiments, the zeolite is maintained below about 500° C. In certain other embodiments, the zeolite is maintained below about 400° C. In yet other embodiments, the zeolite is maintained below about 300° C.

In certain embodiments, the solid acid component of the catalyst can be selected from a non-zeolitic solid acid, such as for example, silica-alumina, sulfated oxides of zirconium, titanium, or tin, sulfated mixed oxides of zirconium, molybdenum, tungsten, etc.

In certain embodiments, the catalyst can include a hydrogenating metal. Exemplary metals include the metals of Group VIB and Group VIIIB of the periodic table, and mixtures thereof. In certain preferred embodiments, the metals include platinum and palladium. In certain embodiments, between 0% and 5% of the metal by weight relative to the solid acid catalyst is present.

In certain embodiments, a catalyst promoter can be included with the catalyst being applied to or incorporated onto the ceramic foam catalyst support. Promoters can operate as co-catalysts and enhance the overall catalytic activity of the selected catalyst without substantially increasing overall catalysis costs. Suitable promoters can be selected from a wide variety of metals, including, but not limited to, cerium, yttrium, lanthanum, praseodymium, neodymium, calcium, magnesium, barium and titanium; and mixtures thereof.

In certain embodiments, the zeolite can have an intermediate pore size, such as for example ZSM-5. While ZSM-5 demonstrates greater activity for ethylene/aromatic alkylation, it may also be used for the alkylation of paraffins by olefins. As used herein, “intermediate pore size” means that the zeolites generally exhibit an effective pore aperture in the range of about 0.5 to 0.65 nm when the molecular sieve is in the H-form. The medium or intermediate pore zeolites are represented by zeolites having the structure of ZSM-5, ZSM-11, ZSM-23, ZSM-35, ZSM-48 and TMA (tetramethylammonium) offretite. Of these, ZSM-5 and ZSM-11 are preferred for functional reasons while ZSM-5 is preferred as being the most readily available on a commercial scale. In certain embodiments, the intermediate pore zeolite has a low Si/Al ratio.

In certain embodiments, regeneration of the catalyst can be achieved by contacting the catalyst particles with an organic solvent stream. In certain embodiments, between about 2 and 10 equivalents of organic solvent can be used to regenerate the catalyst, preferably at least about 5. In certain embodiments, the regeneration can take place at room temperature. Suitable solvents for the regeneration of the catalyst can include a wide variety of organic solvents. In certain embodiments, the solvent is selected from a non-polar solvent saturated fatty hydrocarbon having between 4 and 20 carbon atoms. In certain preferred embodiments, the solvent is a non-polar solvent.

In certain other embodiments, regeneration of the catalyst can include heating the catalyst in the presence of hydrogen.

Alkylation

An isoparaffin and olefin can be combined in a reactor that is charged with the ceramic foam supported catalyst prepared according to the methods described herein.

Useful olefins for alkylation reactions can include lighter olefins from ethylene up to butenes (C₂ to C₄), although alkylation reactions can also be performed with heavier olefins from pentenes up to decanes (C₅ to C₁₀), resulting in products that can generally be incorporated into the gasoline product. Preferably, the olefin has between 2 and 6 carbon atoms. More preferably, the olefin has between 3 and 5 carbon atoms. In general, lighter olefins are more useful in providing a contribution to the octane number.

The olefin can include from 2 to 16 carbon atoms. Exemplary olefins include butene-2, isobutylene, butene-1, propylene, pentenes, ethylene, hexene, octene, and heptene. Preferably, the olefin is a butene.

The present process is advantageous in that it operates particularly well with the lighter olefins, each of which can be readily obtained in a refinery as a by-product of the cracking process. This provides a valuable route for the conversion of this cracking by-product to a desired gasoline product. In addition, because the reactant gases are present from other processes, the alkylation process is attractive from an economic standpoint. For this reason, mixed olefin streams, such as for example, an FCC Off-Gas stream (which can typically include ethylene, propylene and butenes), can also be used. Alkylation using C₃ and C₄ olefin fractions obtained during the cracking process can thus provide an easy route to producing branch chain C₆, C₇ and C₈ products, each of which can be highly desirable as gasoline additives from the view point of boiling point and octane. This process can advantageously be conducted at the refinery site.

Paraffin Feedstock

The other main component in the alkylation process are paraffins (saturated hydrocarbons or alkanes). Light paraffins can be obtained during oil and gas recovery. Natural gas includes both propanes and butanes. Oil includes liquid alkanes, including pentanes and hexanes.

Exemplary paraffins include linear and branched paraffins having between 4 and 12 carbon atoms. In certain embodiments, the paraffins are isoparaffins having between 4 and 8 carbon atoms. In certain other embodiments, the paraffins can include linear and branched paraffins having between 3 and 5 carbon atoms. In other embodiments, the paraffins are selected from isobutane, isopentane, 3-methylhexane, 2-methylhexane 2,3-dimethylbutane and 2,4-dimethylhexane, and mixtures thereof. In certain preferred embodiments, the paraffin is isobutane.

As previously noted, alkylation is the chemical addition of an alkyl group to another molecule to form a larger molecule. Specifically, with respect to the production of gasoline alkylate additives, lighter olefins are protonated to form a carbocations, which then react with paraffins to produce branched alkanes, particularly substituted heptanes, octanes and nonanes. Typically, the alkylation reaction is performed at mild temperatures in a two-phase reaction. In certain embodiments, the alkylation reaction is performed at a temperature of less than 120° C. In certain other embodiments, the alkylation reaction is performed at a temperature between 0° C and 100° C.

The hydrocarbon feedstock undergoing alkylation can be provided to the reaction zone in a continuous stream containing effective amounts of the olefin and paraffin materials. In certain embodiments, an excess of the paraffin reactant is supplied to the reactor to control the reaction equilibrium and minimize both side reactions and the production of undesired side products. The mole ratio of olefin to paraffin can range from 1:1.5 to 1:30, preferably ranging from about 1:3 to 1:15.

One exemplary alkylation reaction is the reaction isobutane (a paraffin) and isobutylene (a C₄ olefin), in the presence of an acid catalyst, to produce isooctane (2,2,4-trimethylpentane). Isooctane is a highly desired gasoline additive, having an octane rating of 100.

The ratio of acid to hydrocarbon feed by volume can be between 0.01:1 and 5:1, depending upon the catalyst employed. Preferably, the ratio is between 0.5:1 and 2:1

In certain embodiments, the catalysts of the present invention can be used in alkylation reactions for the preparation of compounds useful as additives to increase the RON of the gasoline product. The RON is a measure of the anti-knock rating of gasoline and/or gasoline constituents. The higher the RON number, the better the anti-knock rating of the gasoline. Exemplary compounds having a RON of 90 or higher that can be prepared with the ceramic foam supported catalysts of the present invention include, but are not limited to, 2,2-dimethyl butane, 2,3-dimethyl butane, trimethyl butane, 2,3-dimethyl pentane, 2,2,4-trimethyl pentane, 2,2,3-trimethyl pentane, 2,3,4-trimethyl pentane, 2,3,3-trimethyl pentane, and 2,2,5-trimethyl hexane.

The alkylation reaction can be performed in one or more alkylation reactors that can include any type of catalyst bed, including but not limited to, a fixed bed, a fluidized bed, a slurry suspension, or the like.

While the invention has been specifically described with respect to alkylation of saturated hydrocarbons with a light olefin to provide saturated hydrocarbons of higher molecular weight, it is understood that the solid acid catalysts of the present invention can be used in a variety of reactions, including alkylation reactions involving aromatics and higher molecular weight olefins.

Materials can be prepared utilizing the ceramic foam support materials coated with a wide variety of catalyst or zeolite. In general, any hydrocarbon conversion process which is capable of being catalyzed by a zeolite, acid catalyst or metal can be conducted with the catalyst support materials prepared according to this invention. Exemplary catalytic processes which the present materials can be used for include, but are not limited to, cracking, hydrocracking, alkylation, isomerizations, polymerization, reforming, hydrogenation, dehydrogenation, and transalkylation. For example, catalytic cracking processes can be carried out on feedstocks such as gas oils, heavy naphthas, etc., using ceramic foam catalyst support zeolite beta compositions.

### EXAMPLE

Suitable ceramic foams for use in the process are available commercially, such as for example, from Selee Inc., Hi-Tech, Vesuvius and Dytech. The coating of the ceramic foam with an active catalytic material such as zeolite crystals, is common in the art. In situ crystallization of zeolite particles from a precursor sol on various supports is one common coating method known in the art.

### Example 1

A ceramic foam support is provided by known means having an average surface area of 6 m²/mg, a porosity of approximately 0.8, a pore density of approximately 45 pore/inch, and a mean pore diameter of approximately 0.40 mm. The ceramic foam support is in the form of particles having a diameter of approximately 3-6 mm.

A coating that includes zeolite-Y crystals can be applied to the ceramic foam support by washcoating. The coating can include zeolite catalysts having an average size of approximately 0.5 μm in diameter, and a pore size of approximately 1 nm.

As used herein, the terms about and approximately should be interpreted to include any values which are within 5% of the recited value. Furthermore, recitation of the term about and approximately with respect to a range of values should be interpreted to include both the upper and lower end of the recited range.

While the invention has been shown or described in only some of its embodiments, it should be apparent to those skilled in the art that it is not so limited, but is susceptible to various changes without departing from the scope of the invention.

1. A method for producing a gasoline alkylation catalyst comprising the steps of:
providing a ceramic foam support having a surface, the ceramic foam support operable to support particles of solid acid catalyst on the surface of the ceramic foam, the ceramic foam support having a porosity of at least 75% and a pore density of at least 10 pores per inch;
applying a coating to the surface of the ceramic foam support such that particles of the solid acid catalyst adhere to the surface of the ceramic foam support to create the gasoline alkylation catalyst, the coating comprising a carrier fluid and the solid acid catalyst particles, the carrier fluid being suitable to maintain the particles of the solid acid catalyst in a dispersion, the solid acid catalyst having an acidity activity index of at least 1.0.

2. The method of claim 1 wherein the solid acid catalyst is a zeolite.

3. The method of claim 2 wherein the zeolite is selected from zeolite-X, zeolite-Y, zeolite beta, MCM-36 and ITQ-2.

4. The method of claim 2 wherein the zeolite has an average pore diameter of at least 5 Å.

5. The method of claim 1 wherein the solid acid catalyst is applied to the surface of the ceramic foam support by washcoating or impregnation.

6. The method of claim 1 wherein the ceramic foam support is a particle having an average diameter of between 2 and 6 mm.

7. The method of claim 1 wherein applying the solid acid catalyst to the surface of the ceramic foam support further comprises the step of:
removing essentially all of the carrier fluid from the gasoline alkylation catalyst through heating.

8. A gasoline alkylation catalyst comprising:
a ceramic foam support, the ceramic foam support having a porosity of greater than 70%; the ceramic foam support having an interior and an exterior surface, the interior surface defining a tortuous path; and
particles of a solid acid catalyst adhered to the surface of the ceramic foam support; the particle of solid acid catalyst defining a particle size distribution; the, the solid acid catalyst having an acid activity index of greater than 1.0; the gasoline alkylation catalyst being operable to convert alkane and olefin feed into an alkylate product;
wherein the ceramic foam support comprises particles having a particle size distribution generally in the range of 3 mm to 6 mm.

9. The method of claim 8 wherein the solid acid catalyst is selected from zeolite-X, zeolite-Y, zeolite beta, MCM-36 and ITQ-2.

10. The method of claim 8 wherein the solid acid catalyst is a zeolite having an average pore diameter of at least 5 Å.

11. A method for alkylating an alkane with an olefin comprising:
introducing an alkane and olefin into a reaction zone, wherein said reaction zone comprises an alkylation catalyst to produce an alkylate product, wherein said alkylation catalyst comprising a solid acid catalyst on a ceramic foam particle support, and said ceramic foam particle support having an average diameter of between 2 and 6 mm; and
collecting a product stream comprising the alkylate product;
wherein said ceramic foam support has a porosity of at least 70%;
wherein the alkane comprises between 3 and 8 carbon atoms, and wherein the olefin comprises between 2 and 8 carbon atoms;

12. The method of claim 11 wherein the solid acid catalyst is selected from zeolite-X, zeolite-Y, zeolite beta, MCM-36 and ITQ-2.

13. The method of claim 11 wherein the solid acid catalyst has an acidity activity index of greater than 1.

14. The method of claim 11 further comprising regenerating the catalyst.

15. The method of claim 11 wherein the alkane is selected from isobutane, isopentane, 2,3-dimethylbutane, 2-methylhexane and 2,4-dimethylhexane.

16. The method of claim 11 wherein the olefin is selected from ethylene, propylene, butylene, and isobutylene.

17. The method of claim 11 wherein the mole ratio of alkane to olefin is between 1.5:1 and 15:1.
